(19) United States
(12) Patent Application Publication
(10) Pub. No.: US 2026/0221255 A1
(43) Pub. Date: Jul. 30, 2026

(54) The Credibles Method

(71) Applicant: Demi Baez, Houston, TX (US)

(72) Inventor: Demi Baez, Houston, TX (US)

(21) Appl. No.: 19/037,985

(22) Filed: Jan. 27, 2025

(51) Int. Cl. G16H 20/10; G16H 10/20

(52) U.S. Cl. CPC G16H 20/10; G16H 10/20

(57) ABSTRACT

The Credibles method is a method for calculating the overall THC potency of hemp or cannabis-infused edible products using qualitative consumer feedback. The system gathers user-reported data on personal tolerance, serving size, and perceived product strength, and converts this into a numeric potency value using a pre-defined array table. The output is a front-end potency percentage (P %) to guide dosage and product selection. This method enhances consumer confidence, encourages responsible consumption, and provides a real-time, personalized understanding of product strength.

### BACKGROUND

Determining a product's overall THC potency will address the growing demand for precise dosing and consistent quality. This method offers a general understanding of the product's intensity prior to consumption, thereby enhancing consumer experience and satisfaction.

Currently, there is no system available that dynamically quantifies qualitative product feedback and generates a front-end overall potency percentage. Traditional sites have attempted to measure a product's potency through qualitative reviews and static data, lacking the ability to integrate real-time user experiences. Furthermore, current systems rarely offer a personalized approach that considers the subjective nature of individual experiences with hemp-derived or cannabis-infused edibles. This limitation highlights the need for a more data-friendly and consumer-centric solution.

### SUMMARY

This Credibles method determines a product's overall potency strength by converting qualitative user feedback into a dynamic front-end potency percentage. It factors in three qualitative questions that pertain to the user's experience with the product. The responses are then quantified using a 0-5 array value and a percentage calculation.

### FIGURE DESCRIPTIONS

FIG. 1: User feedback questions factored in to find Potency Value (PV).

FIG. 2: Value array table illustrating how (PV) is determined.

FIG. 3: A front-end visual of a calculated Potency Percentage (P %)

FIG. 4: Consumer dosing guide to determine a dosage based on percentage results and consumer tolerance level

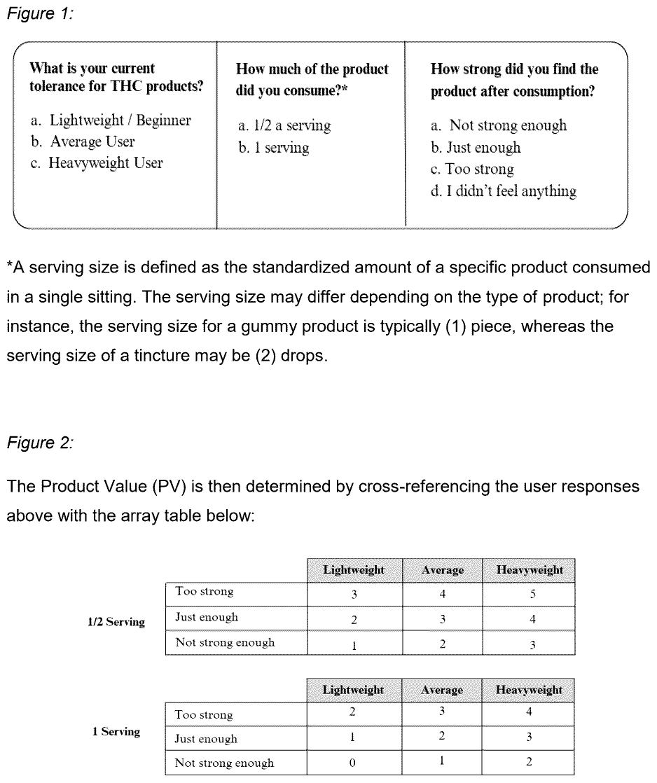

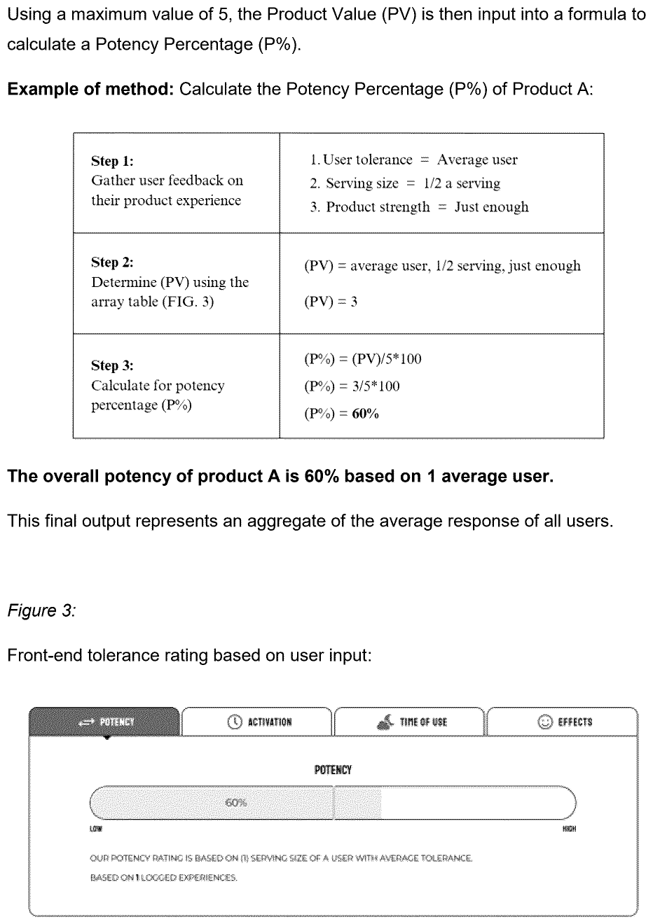

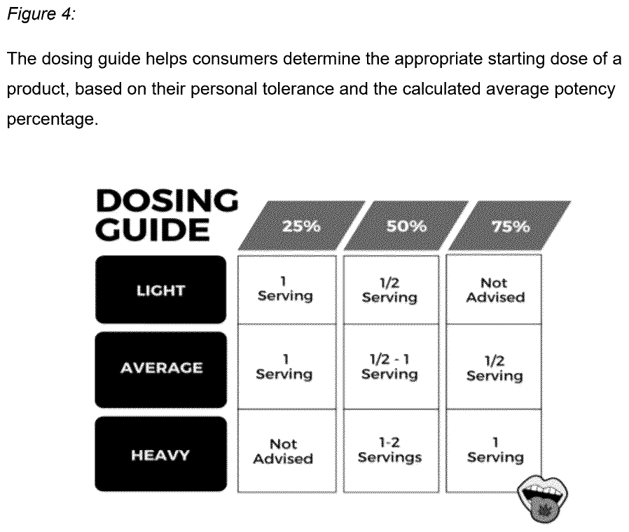

### DETAILED DESCRIPTION OF INVENTION

The Credibles method comprises the following components:
User Inputs:
   Tolerance Level: Lightweight, Average, or Heavyweight
   Serving Size Consumed: ½ serving or 1 serving
   Perceived Product Strength: Not strong enough, Just enough, or Too strong
Data Collection
   Responses are gathered via an anonymous survey system tied to each product page.
   A standardized serving size is defined per product category (e.g., 1 gummy or 2 tincture drops).
   Potency Value (PV) Assignment
   Responses are cross-referenced with a 0-5 value array table.
   Based on P % and personal tolerance, users are guided to select an appropriate starting dose.
Potency Percentage (P %) Calculation

$$P \% = ( PV \div 5 ) \times 100 \qquad Formula$$

Example:
   Tolerance=Average
   Serving=½
   Perceived strength=Just enough

$$PV = 3 \to P \% = 60 \%$$

Output.
   The calculated P % represents the average product intensity for consumers with similar profiles.
   A visual potency rating is displayed to guide future shoppers.
Consumer Dosing Guide
   Based on P % and personal tolerance, users are guided to select an appropriate starting dose.

### 1. Embodiments

This method may be extended or adapted with:
Additional consumption options (¼ and ¾ servings) for high-potency items.
Expanded product categories (e.g., beverages, baked goods, psychedelic edibles).
\Inclusion of context factors (e.g., state of appetite at time of use).

1. A method for determining the overall potency of a hemp- or cannabis-infused edible product, comprising:
Receiving user input data, including tolerance level, serving size consumed, and perceived product strength.
Quantifying qualitative feedback using a defined value array table to generate a Product Value (PV).
Calculating a Potency Percentage (P %) by applying a mathematical formula:
$$( PV \div 5 ) \times 100.$$
Displaying a dynamic front-end potency percentage for product representation.
Providing a dosing guide based on P % and user tolerance.
